(19) United States
(12) Patent Application Publication
(10) Pub. No.: US 2018/0321125 A1
(43) Pub. Date: Nov. 8, 2018

(54) GAS SENSOR

(71) Applicant: Robert Bosch GmbH, Stuttgart (DE)

(72) Inventors: Christopher Holzknecht, Stuttgart (DE); Karel Vacha, Mirkovice (CZ); Martin Buchholz, Bietigheim-Bissingen (DE); Sebastian Schulte Am Huelse, Stuttgart (DE); Simon Rentschler, Schwaikheim (DE); Alan Celic, Stuttgart (DE)

(21) Appl. No.: 15/775,081

(22) PCT Filed: Oct. 26, 2016
(86) PCT No.: PCT/EP2016/075773

§ 371 Date: May 10, 2018

(30) Foreign Application Priority Data
Dec. 7, 2015 (DE) 10 2015 224 460.1

(51) Int. Cl. G01N 15/06; G01M 15/10; G01N 33/00

(52) U.S. Cl. CPC G01N 15/06; G01N 33/0009; G01M 15/102

(57) ABSTRACT

A gas sensor for determining at least one constituent or at least one property of a measuring gas, in particular, of an exhaust gas of an internal combustion engine, including a sensor element. An outer protective tube of the gas sensor includes at least one inlet opening for measuring gas to enter into an outer chamber, the at least one inlet opening includes at least one swirl element for forming a vortex about the longitudinal direction in the outer chamber. The outer chamber protrudes distally beyond a housing in the longitudinal direction by an outer chamber longitudinal extent and an inner chamber protrudes distally beyond the housing in the longitudinal direction by an inner chamber longitudinal extent, and the outer chamber longitudinal extent being at least twice as large as the inner chamber longitudinal extent.

### BACKGROUND INFORMATION

A gas sensor for determining at least one constituent or at least one property of a measuring gas is available in the related art.

The gas sensor described in European Patent No. EP 0 978 721 B1 includes a detection element having a front section, a detection section which is formed on the front section of the detection element; and a protector that covers the detection section, the protector including a first section and a second section situated radially outside the first section, the first section of which includes a sidewall having a first gas inlet, the sidewall including an axial front end and a tapering section, the tapering section thereof being formed from the axial front end of the sidewall, at least one second side gas inlet being formed in a side wall section of the second section and a first gas outlet being formed in the first section, the at least one second side gas inlet being situated at a point radially opposite the tapering section; and a second gas outlet being formed in the second section, the first gas outlet being formed in a front end surface of the second section, and the second gas outlet being formed in a front end surface of the second section, the tapering section of the first section being in the form of a truncated cone, which is connected to the front end of a cylindrical body.

### SUMMARY

An object of the present invention is to provide a gas sensor, which simultaneously preferably effectively implements multiple properties that were previously considered to conflict with one another.

Thus, the sensor function and the heatability, as well as the dynamics of the gas sensor are to be independent of its orientation about its longitudinal axis, when it is exposed to a lateral measuring gas flow. The gas sensor is also to be robust against particles contained in the measuring gas, such as water drops and/or soot particles. At the same time, however, the gas sensor should also include high dynamics, i.e., in the case of changes in the concentration of the constituent of the measuring gas or in the case of changes of the property of the measuring gas, it should very quickly provide a correspondingly modified signal.

An example gas sensor according to the present invention may accomplish this.

An example gas sensor according to the present invention includes a housing, which in turn, for example, includes a thread and an external hex, so that the gas sensor is screwable with its distal end into a receiving socket of an exhaust gas tract of an internal combustion engine.

A sensor element is installed in the housing. It may be, for example, a planar or rod-shaped, sintered, ceramic sensor element of an exhaust gas sensor, for example, of an oxygen sensor or of a NOx sensor or of a particle sensor, which is known, in principle, from the related art. The housing includes, for example, an internal bore, in which the sensor element is retained by a sealing device, which is made for example, of steatite and/or of boron nitride. The sensor element protrudes in both longitudinal directions beyond the sealing device and beyond the housing, for example.

The sensor element includes a gas-sensitive end section, which includes, for example, at least one electrode of an electrochemical cell or an interdigital electrode. This gas-sensitive end section protrudes distally from the housing and from the sealing device in the longitudinal direction of the gas sensor and is therefore exposed to the measuring gas.

The gas-sensitive end section of the sensor element is covered with a protective tube module that is fastened to the housing, so that the measuring gas does not interact directly with the sensor element, but in a manner which is defined by the geometry and configuration of the protective tube module. The protective tube module may, for example, be fastened to the housing by a peripheral weld.

The protective tube module includes an inner protective tube and an outer protective tube, the inner protective tube enclosing the gas-sensitive end section of the sensor element at a radial distance and axial distance, and further enclosing an inner chamber, and the outer protective tube enclosing the inner protective tube, and further enclosing an outer chamber formed between the outer protective tube and the inner protective tube.

The outer protective tube includes at least one inlet opening. The at least one inlet opening is understood within the scope of the present invention to mean the one inlet opening if there is precisely only one inlet opening, alternatively, it is understood to mean all inlet openings if there are multiple inlet openings.

The outer protective tube further includes at least one outlet opening. The at least one outlet opening is understood within the scope of the present invention to mean the one outlet opening if there is precisely only one outlet opening, alternatively, it is understood to mean all outlet openings if there are multiple outlet openings.

The inner protective tube also includes at least one inlet opening and at least one outlet opening. This is to be understood in the sense of that which was explained above for the at least one inlet opening and for the at least one outlet opening of the outer protective tube.

To establish whether an opening of the outer protective tube is an inlet opening or an outlet opening, it may be assumed, in particular, that the at least one outlet opening is situated in the longitudinal direction distally to the at least one inlet opening and/or that the at least one outlet opening is situated in the radial direction within the at least one inlet opening. As explained, the at least one inlet opening and/or the at least one outlet opening may each include multiple openings. The indicated relation is then applicable for each inlet opening in relation to each outlet opening.

To establish whether an opening of the inner protective tube is an inlet opening or an outlet opening, the same applies, in particular, analogously.

The classification of the openings into inlet openings and outlet openings made in the preceding paragraphs reflects, in particular, which flows form in a gas sensor according to the present invention, which is exposed to an outer lateral gas flow, the flow velocity of which is greater in the area of the distal end of the gas sensor than in a more proximal area of the gas sensor. This applies to a gas sensor, for example, which is properly screwed into the receiving socket of an exhaust gas tract of an internal combustion engine, i.e. without intersecting a central axis of a line of the exhaust gas tract.

Thus, during operation of the exhaust gas sensor according to the present invention, measuring gas, in particular, enters through the at least one inlet opening of the outer protective tube into the protective tube module and into the outer chamber.

The at least one inlet opening of the outer protective tube includes a swirl element, in the event of multiple inlet openings of the outer protective tube, each inlet opening of the outer protective tube, in particular, includes a swirl element, so that a vortex is formed in the outer chamber about the longitudinal axis of the gas sensor. Thus, at least a portion of the measuring gas, which may also be referred to as the main flow, following the vortex, arrives at the at least one outlet opening of the outer protective tube, where it exits the protective tube module. Massive particles potentially present in the exhaust gas, such as water drops and/or soot, are transported in this way along the outer protective tube in the distal direction to the outlet opening of the outer protective tube, where they are removed from the protective tube module, without ever interacting in a potentially harmful manner with the gas-sensitive end section of the sensor element.

Moreover, the effect of the vortex formed about the longitudinal axis of the gas sensor is that a static pressure is lower in its interior than in its outer areas. In the present case, this produces, in particular, a pressure gradient according to a relative overpressure in the area of the inlet openings of the inner protective tube and according to a relative negative pressure in the area of the outlet opening of the inner protective tube. The pressure gradient drives, in particular, a corresponding flow through the inner protective tube, which transports measuring gas to the gas-sensitive end section of the sensor element. This flow through the inner protective tube represents, in particular, a bypass flow to the main flow, which branches off from the main flow in the outer chamber, strikes the gas-sensitive end section of the sensor element after a, in particular, comparatively short flow passage and, after flowing through the inner chamber and re-entering the outer chamber, in particular, rejoins the main flow.

Thus, according to the present invention, it has been found that the formation of the vortex in the outer chamber is able to produce the advantageous effects described at the outset.

According to the present invention, it has also been found that these effects appear all the more so, the more undisturbed the vortex is able to form in the outer chamber, i.e., the less the vortex flow is slowed in the outer chamber by friction against the protective tube module or by flow obstructions.

It is therefore provided according to the present invention that the outer chamber of the housing protrudes distally in the longitudinal direction by an outer chamber longitudinal extent, and that the inner chamber of the housing protrudes distally in the longitudinal direction by an inner chamber longitudinal extent, and that the outer chamber longitudinal extent is at least twice as large as the inner chamber longitudinal extent. This results in a correspondingly large extension of the outer chamber, in which the vortex flow may be formed freely and largely unimpeded, undisturbed by the inner protective tube.

By contrast, in gas sensors known from the related art, which lack the features essential to the present invention, and in which the at least one side gas inlet is situated at one point radially opposite the tapering section, the result is essentially merely the formation of a rotating gap flow with correspondingly high frictional losses.

In other words, it is therefore provided according to the present invention, in particular, that the vortex formed in the outer chamber drives a flow through the inner protective tube via the formation of a static pressure gradient between the inlet opening of the inner protective tube and the outlet opening of the inner protective tube, and the vortex formed in the outer chamber is formed largely and predominantly in a chamber area of the outer chamber, which lies completely opposite the housing in the longitudinal direction, as viewed from the inner chamber.

The formation of the vortex is assisted by the configuration of the openings of the inner protective tube and of the outer protective tube when all openings of the outer protective tube, i.e., the at least one inlet opening and the at least one outlet opening of the outer protective tube, are situated distally of all openings of the inner protective tube, i.e., distally to the at least one inlet opening and distally to the at least one outlet opening of the inner protective tube.

The inlet openings of the outer protective tube may be in the form of a plurality of inlet openings, which may be situated at the same height in the longitudinal direction on an outer surface of the outer protective tube. In this regard, they may form a perforated collar, on which the openings are situated equidistantly from their closest neighbor. Six or eight openings may be provided, for example. It may be provided that each inlet opening includes a swirl element, in particular, a swirl flap.

The swirl flaps may be made by producing a straight cut in the outer protective tube and subsequently pushing in or pushing out the area of the outer protective tube adjacent to the cut. The swirl flaps are preferably shaped in such a way that a flow entering into the outer protective tube contacts essentially merely tangentially the outer protective tube and a radial flow component is merely small. This is achievable by shaping the swirl flaps convexly in a first area spaced apart from the cut, as viewed from the outside, and concavely in a second area facing the cut.

The formation of a strong flow, in particular, a strong main flow, in the outer protective tube may be assisted in that the swirl flap or the swirl flaps are oriented in the distal longitudinal direction, i.e., they impart a velocity component to the entering flow in this direction. The swirl flap or the swirl flaps may, for example, be oriented in such a way that the direction of the entering flow bisects the angle between the tangential direction and the axial direction (45°).

It may be advantageous for the inner protective tube to be situated closely to the sensor element. In particular, in the case of a heated sensor element and, by comparison, a cool exhaust gas, the inner protective tube is intensely heated in this way, subsequently resulting in a reduced input of soot into the inner protective tube due to the thermophoretic effect. The narrow contact may be quantitatively expressed by the fact that the distance in the longitudinal direction of the gas-sensitive end section in the inner protective tube is no more than 15% of the outer chamber longitudinal extent.

The formation of the vortex in the outer protective tube may be further improved if the outer protective tube is curved and tapers spherically in the process in the area between the at least one inlet opening of the outer protective tube and the at least one outlet opening of the outer protective tube.

In one refinement, it may be provided that at the distal end of the outer protective tube, the transition of the outer protective tube into the outlet opening of the outer protective tube is curved, leaving the outer protective tube without an end surface in the proper sense at its distal end. The effect of this step is that massive particles, for example, soot particles or drops that are pushed along the outer protective tube in the distal direction, are able to exit the protective tube module through the outlet opening of the outer protective tube with no further resistance.

Refinements of the present invention are described herein in the context of an exemplary embodiment.

### BRIEF DESCRIPTION OF THE DRAWINGS

The present invention is explained in greater detail in the description below with reference to the exemplary embodiment depicted in the figures.

FIG. 1 shows a detail of a longitudinal section through an example gas sensor according to the present invention.

FIG. 2 shows the example gas sensor according to the present invention when properly installed in an exhaust gas line.

FIG. 3 schematically shows the gas flows forming in the protective tube module of the example gas sensor from FIG. 1 when properly installed according to FIG. 2.

### DETAILED DESCRIPTION OF EXAMPLE EMBODIMENTS

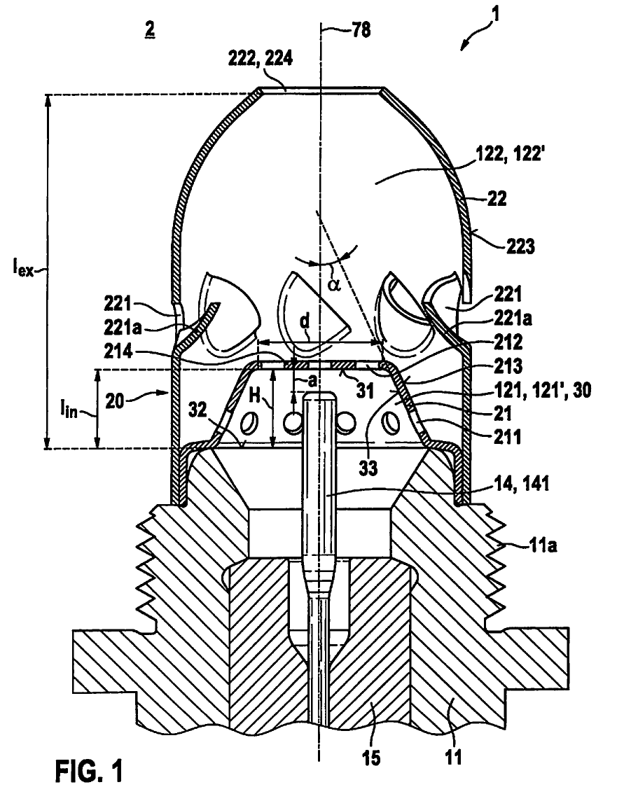

FIG. 1 shows a gas sensor according to the present invention, for example, a broadband lambda sensor. The gas sensor includes a housing 11. A ceramic sensor element 14 is fastened in housing 11 by a seal 15, which is made of steatite and/or boron nitride, for example. A gas-sensitive end section 141 of sensor element 14 projects distally from seal 15 and from housing 11 in a longitudinal direction 78 of gas sensor 1 and is exposed to a measuring gas. A protective tube module 20 is fastened to housing 11 by a weld, so that it covers the gas-sensitive end section 141.

Protective tube module 20 includes an inner protective tube 21 and an outer protective tube 22.

Inner protective tube 21 encloses gas-sensitive end section 141 at a radial and axial distance. Thus, an inner chamber 121 is formed between inner protective tube 21 and housing 11, in which gas-sensitive end section 141 is located. Distance a between sensor element 14 and inner protective tube 21 in the axial direction is only 1 mm, for example, so that when sensor element 14 is heated, inner protective tube 21 is also heated, which has the advantage that a deposition of particles, for example, soot particles, on inner protective tube 21 or on sensor element 14 is suppressed as a result of thermophoresis.

Inner chamber 121 protrudes distally beyond housing 11 in longitudinal direction 78 by an inner chamber longitudinal extent lᵢₙ, which in this example is 4 mm. Chamber area 121′ of inner chamber 121 protruding distally beyond housing 11 in longitudinal direction 78 has the shape of a straight truncated cone 30 tapering in the distal direction. Cover area 31 of truncated cone 30 is approximately half as large as base area 32 of truncated cone 30. Height H of truncated cone 30 is smaller than diameter d of cover area 31. Outer surface area 33 of truncated cone 30 is inclined at an angle α toward longitudinal direction 78, which in this example is 23°.

Inner protective tube 21 includes a perforated collar on its outer surface 213 of, for example, 10 inlet openings 211, which are situated at the same height and spaced equidistantly apart from one another, and which are overtopped distally by gas-sensitive end section 141 in longitudinal direction 78. Inner protective tube 21 includes a plurality of outlet openings 212 on its end surface, which forms its distal end 214.

Outer protective tube 22 encloses inner protective tube 21, so that an outer chamber 122 is formed in the interior of protective tube module 20 between outer protective tube 22 and inner protective tube 21. Outer protective tube 22 includes 8 inlet openings 221, which are situated at the same axial height on outer surface 223 of outer protective tube 22, and equidistant from one another on a perforated collar. Inlet openings 221 are situated in the longitudinal direction distally to inner protective tube 21.

Inlet openings 221 include swirl elements 221a, which are directed below 45° relative to the tangent at the outer protective tube diagonally in the distal direction, i.e., away from housing 11. The swirl elements are made, for example, by producing a straight cut in outer protective tube 22 and subsequently pushing in the area of outer protective tube 22 adjacent to the cut. Swirl flaps 221a are convex in the area spaced apart from the cut and are concave in the area facing the cut, in each case from the perspective outside of outer protective tube 22 onto swirl flaps 221a.

Outer protective tube 22 includes a single outlet opening 222, which is situated centrally at distal end 224 of outer protective tube 22. Outer protective tube 22 is curved in the distal direction between inlet openings 221 of outer protective tube 22 and outlet opening 222 of outer protective tube 22, and tapers spherically in the process. At its distal end 224, outer protective tube 22 merges into the single outlet opening 222 of outer protective tube 22, leaving outer protective tube 22 without an end surface in the proper sense on its distal end 224.

Outer chamber 122 protrudes beyond housing 11 in longitudinal direction 78 by an outer chamber longitudinal extent lₑₓ, which in the example is 15 mm.

Gas sensors 1 of the type in question are installed, in particular, properly in an exhaust gas line 2, for example, of an internal combustion engine, specifically in such a way that exhaust gas flows laterally against them, i.e., perpendicularly to longitudinal direction 78 of the gas sensor. Deviations from a precisely perpendicular flow, for example, by up to 8° are also possible and are generally well tolerable in the design provided.

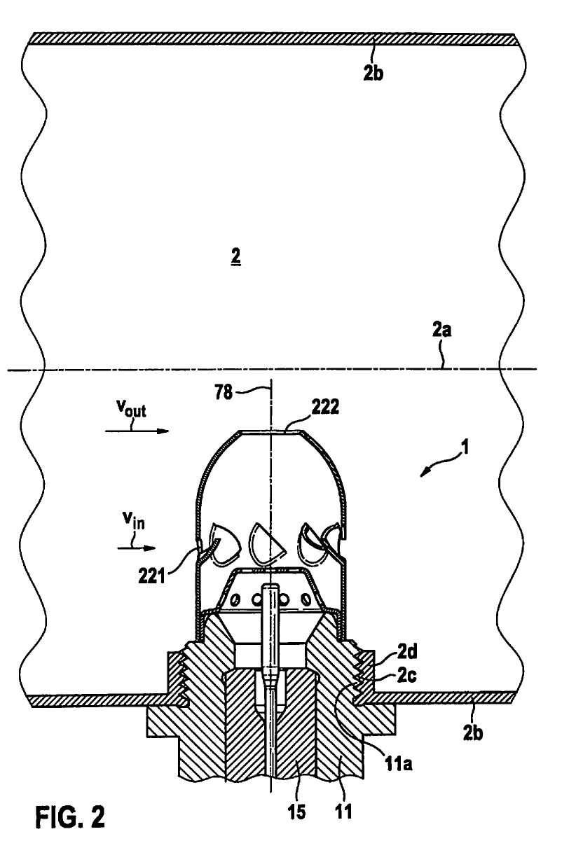

In this case, a flow velocity vₒᵤₜ in the area of outlet opening 222 of outer protective tube 22 formed as a face hole of exhaust gas sensor 1 is greater than a flow velocity vᵢₙ in the area of inlet openings 221 of outer protective tube 22. Such flow conditions are present, for example, if gas sensor 1, as in FIG. 2, is screwed with its thread 11a into its mating thread 2c of a receiving socket 2d integrated in wall 2b of exhaust gas line 2, without overlapping a center axis 2a of exhaust gas line 2 in the process. It is preferred that the distal end of gas sensor 1 is directed downwardly, so that a penetration of drops potentially contained in the measuring gas is gravitationally reduced.

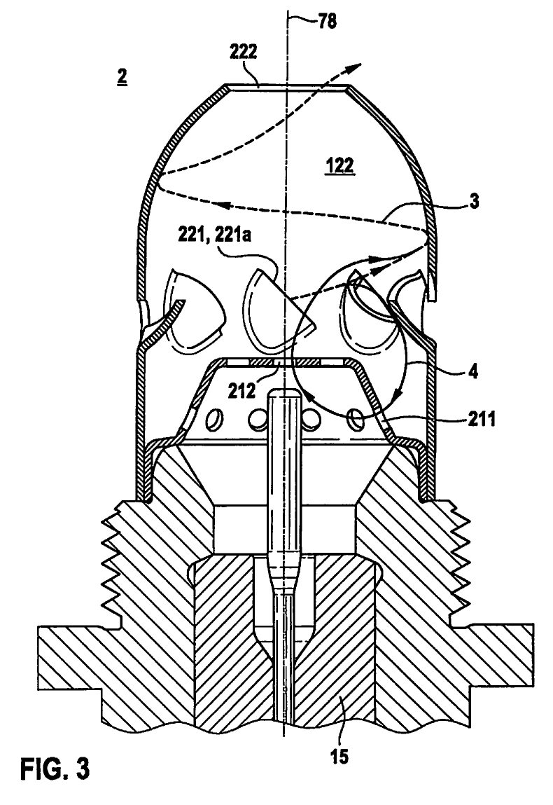

As a result of the different flow velocities vₒᵤₜ, vᵢₙ in the area of inlet openings 221 and outlet openings 222 of outer protective tube 22, a gradient of the static pressure forms inside outer protective tube 22, which drives a flow through outer protective tube 22 from inlet openings 221 to outlet openings 222, see FIG. 3.

Due to the design of inlet openings 221 with swirl flaps 221a, the measuring gas enters into outer chamber 122 with a tangential velocity component. Thus, a vortex of the measuring gas on the whole is formed about longitudinal axis 78 of the gas sensor. The vortex is plotted with dashed lines in FIG. 3. In the interaction of the vortex with the flow through outer protective tube 22, the measuring gas flows in helical-shaped paths along a main flow 3, one of which is plotted in FIG. 3, from inlet openings 221 to outlet opening 222 of the outer protective tube. The vortex in this case is formed largely and predominantly in a chamber area 122′ of outer chamber 122, which is situated completely distally to inner chamber 121. Thus, the formation of the vortex is largely undisturbed by inner protective tube 21.

Massive particles, in particular, such as soot particles and/or water drops thus pass through outer protective tube 22, driven by main flow 3 and its inertia in a helical path along the inner surface of outer protective tube 22. They exit outlet opening 222 of outer protective tube 22 at distal end 224 of outer protective tube 22, without ever interacting in a potentially harmful manner with gas-sensitive end section 141 of sensor element 14.

The formation of the vortex in outer chamber 122 has the further effect that a static pressure in the interior of the outer chamber close to longitudinal axis 78 of the gas sensor is lower than a static pressure in the outer area.

In this way, a relative overpressure forms in the area of inlet openings 211 of inner protective tube 21 and a relative negative pressure forms in the area of outlet opening 212 of inner protective tube 21. The result is a flow through of inner protective tube 21 by a bypass flow 4, which is branched off from main flow 3. This bypass flow 4 strikes gas-sensitive end section 141 of sensor element 14 in inner chamber 121 after a comparatively short flow passage, i.e., after a very short period of time. Subsequently, bypass flow 4 exits outlet opening 212 of inner protective tube 21 back into outer chamber 122 and rejoins main flow 3.

Due to the relatively strong deflection when bypass flow 4 branches off from main flow 3, massive particles, such as water drops and/or soot particles largely follow main flow 3. Thus, they do not reach gas-sensitive end section 141 of sensor element 14 in a potentially harmful manner.

1-15. (canceled)

16. A gas sensor for determining at least one constituent or at least one property of a measuring gas of an exhaust gas of an internal combustion engine, comprising:
a sensor element installed in a housing, the sensor element including a gas-sensitive end section that protrudes distally from the housing along a longitudinal direction of the gas sensor and which is exposed to the measuring gas;
a protective tube module which covers the gas-sensitive end section and which is fastened to the housing, the protective tube module including an inner protective tube that encloses the gas-sensitive end section and is fastened to the housing, the protective tube module including an inner protective tube which encloses the end section at a radial distance and axial distance, so that an inner chamber is formed between the housing and the inner protective tube, in which the gas-sensitive end section is located, the protective tube module including an outer protective tube, which encloses the inner protective tube, so that an outer chamber is formed in an interior of the protective tube module between the outer protective tube and the inner protective tube, the outer protective tube including at least one inlet opening for measuring gas to enter into the outer chamber, the at least one inlet opening of the outer protective tube including at least one swirl element for forming a vortex about the longitudinal direction in the outer chamber, and the outer protective tube also including at least one outlet opening for measuring gas to exit the protective tube module from the outer chamber, the inner protective tube including at least one inlet opening for measuring gas to enter the inner chamber from the outer chamber, and at least one outlet opening for measuring gas to exit from the inner chamber into the outer chamber;
wherein the outer chamber protrudes distally beyond the housing in the longitudinal direction by an outer chamber longitudinal extent and the inner chamber protrudes distally beyond the housing in the longitudinal direction by an inner chamber longitudinal extent, and the outer chamber longitudinal extent is at least twice as large as the inner chamber longitudinal extent.

17. The gas sensor as recited in claim 16, wherein the vortex formed in the outer chamber drives a flow through the inner protective tube via the formation of a static pressure gradient between the inlet opening of the inner protective tube and the outlet opening of the inner protective tube, and the vortex formed in the outer chamber being formed completely or largely and predominantly in a chamber area of the outer chamber, which is situated completely opposite the housing in the longitudinal direction, as viewed from a perspective of the inner chamber.

18. The gas sensor as recited in claim 16, wherein the at least one inlet opening of the inner protective tube and the at least one outlet opening of the inner protective tube are all situated proximal to all of the at least one inlet opening of the outer protective tube and of the at least one outlet opening of the outer protective tube.

19. The gas sensor as recited in claim 16, wherein the at least one inlet opening of the outer protective tube is formed by a plurality of inlet openings situated on the outer surface of the outer protective tube, at least one swirl element, respectively, being situated at each of the inlet openings.

20. The gas sensor as recited in claim 16, wherein the plurality of inlet openings located on the outer surface of the outer protective tube are situated on a side of the inner protective tube which faces away from the housing in the longitudinal direction.

21. The gas sensor as recited in claim 16, wherein the at least one outlet opening of the outer protective tube is a single outlet opening, which is situated centrally at the distal end of the outer protective tube.

22. The gas sensor as recited in claim 16, wherein the at least one inlet opening of the inner protective tube is formed by a plurality of inlet openings located on an outer surface of the inner protective tube.

23. The gas sensor as recited in claim 16, wherein the at least one outlet opening of the inner protective tube is situated at the distal end of the inner protective tube, multiple outlet openings of the inner protective tube being situated at the distal end of the inner protective tube.

24. The gas sensor as recited in claim 16, wherein the at least one inlet opening of the inner protective tube is formed by a plurality of inlet openings of the inner protective tube, which are located at the same height in the longitudinal direction and are each equidistant from their closest neighbor.

25. The gas sensor as recited in claim 16, wherein the outer protective tube is curved and tapers spherically in a distal direction in an area between the at least one inlet opening and the at least one outlet opening.

26. The gas sensor as recited in claim 16, wherein, at a distal end of the outer protective tube, the outer protective tube merges into the outlet opening of the outer protective tube in a curved manner, so that the outer protective tube has no end surface at its distal end.

27. The gas sensor as recited in claim 16, wherein the gas-sensitive end section protrudes distally beyond the at least one inlet opening of the inner protective tube in the longitudinal direction.

28. The gas sensor as recited in claim 16, wherein a distance in the longitudinal direction of the gas-sensitive end section to the inner protective tube a is not more than 15% of the outer chamber longitudinal extent.

29. The gas sensor as recited in claim 16, wherein the chamber area of the inner chamber protruding distally beyond the housing in the longitudinal direction has the shape of a straight truncated cone tapering in a distal direction, at least one of: (i) a cover area of the truncated cone being approximately half as large as a base area of the truncated cone, (ii) a height of the truncated cone being smaller than a diameter of the cover area, and (iii) an outer surface of the truncated cone being inclined by 15° to 30° toward the longitudinal direction.

30. The gas sensor as recited in claim 16, wherein the gas sensor is designed in such a way that with proper installation of the gas sensor in a flow-through exhaust gas line in the interior of the protective tube module, a main flow is formed, which leads from the inlet opening of the outer protective tube while forming a vortex about the longitudinal direction of the gas sensor, through the outer chamber to the outlet opening of the outer protective tube, and further, a bypass flow branching off from the main flow, which passes through the inlet opening of the inner protective tube to the gas-sensitive end section and from there to the outlet opening of the inner protective tube, and subsequently rejoins the main flow in the outer chamber.
